(19) United States
(12) Patent Application Publication
(10) Pub. No.: US 2010/0234829 A1
(43) Pub. Date: Sep. 16, 2010

(54) DISPENSER HEAD FOR NASAL CLEANSING

(76) Inventor: Wayne Jeffery Perry, Schenectady, NY (US)

Correspondence Address: SUZANNAH K. SUNDBY;SMITH, GAMBRELL & RUSSEL, LLP, 1130 Connecticut Avenue, NW, Suite 1130, WASHINGTON, DC, 20036, US

(21) Appl. No.: 12/634,365

(22) Filed: Dec. 9, 2009

Related U.S. Application Data
(60) Provisional application No. 61/121,119, filed on Dec. 9, 2008.

(51) Int. Cl. A61M 3/02

(52) U.S. Cl. 604/514; 604/257

(57) ABSTRACT

An embodiment of the invention is a nasal cleansing top or dispenser head having a curved shape that tapers from at or near a base portion of the head to a tip portion of the head. The dispenser head further includes an opening or outlet disposed on a spout end at the tip through which a cleansing liquid such as, for example, saline can be dispensed. The dispenser head can be removably attached to a vessel, such as a nasal rinse bottle, allowing the user to convert the vessel into a neti pot by allowing the vessel to be tilted along with the user's head.

### CROSS-REFERENCE TO RELATED APPLICATIONS

The present invention relates to, and is entitled to the benefit of the earlier filing date and priority of, U.S. application No. 61/121,119 filed Dec. 9, 2008, which is herein incorporated by reference as if fully set forth.

### FIELD OF THE INVENTION

The present invention relates to a nasal cleansing dispenser head, and more particularly, to a nasal cleansing dispenser head that can be fitted to allow a vessel, such as a nasal rinse bottle, to be converted into a neti pot.

### BACKGROUND

Rinsing the nasal and sinus cavities with saline solution for the purpose of improving airflow and relieving nasal congestion has been recommended by doctors and practiced widely across numerous cultures for hundreds of years. The term Jala neti translated literally means nasal cleansing with water in Sanskrit.

Clinical studies demonstrate that a regular regimen of nasal cleansing is an effective therapy to relieve sinusitis, rhinosinusitis, allergies, upper respiratory tract infections, allergic rhinitis, rhinorrhea (runny nose), and an effective preventative therapy for snoring and the common cold.

Such clinical studies indicate that saline nasal cleansing is a highly effective, minimally invasive method of treating the symptoms of sinus and nasal related disease by delivering to the nasal cavity a streaming dose of saline.

The three methods by which the nasal cavity can be cleansed are: gravity, pressure, and suction.

Pressure-based devices can impart painful and unpleasant overpressure in the nasal passageway and the middle ear via the Eustachian tube.

Pressure-based nasal rinse bottles often employ a squeeze-type dispenser with a dip tube. A drawback to such dispensing means exists in that many individuals are unable to perform nasal cleansing in a vertical direction, and the dip tube means are disadvantageously difficult to operate in anything but a vertical direction.

Given the problems caused by a pressure-based nasal spray bottle with a dip tube within the bottle, the awkwardness in use caused by operating the typical nasal spray bottle by gravity is a further drawback.

Gravity-based devices, for example the neti pot, allow for the flow of the rinsing solution into the nasal cavity by tilting ones head to the side, such that the two nostrils are situated on different planes. The user inserts a spout into the upper nostril and pours the cleansing solution, contained in the reservoir pot, into the nostril. On the basis of gravity, the saline flows in an open circuit around the posterior margin of the nasal septum and out the lower nostril.

The gentler pressure of gravity-based devices such as a neti pot can be advantageous for people who suffer ear discomfort.

Many neti pot users incorporate nasal cleansing into their daily cleansing routine. Much like a toothbrush, these users take their neti pot wherever they travel. Therefore, large, heavy or fragile (e.g. breakable ceramic, glass, etc.) neti pots are disadvantageous, and difficult to store and transport.

In view of the above, it is desirable to provide a convenient dispenser head that can easily convert a vessel and/or rinse bottle, including, but not limited to, a nasal rinse bottle, into a neti pot type apparatus and overcome the disadvantages of a conventional nasal rinse bottle.

### SUMMARY OF THE INVENTION

Responsive to the foregoing challenges, Applicant has developed a novel nasal cleansing dispenser head, and more particularly, a nasal cleansing dispenser head that can be fitted to allow a vessel, such as a nasal rinse bottle, to be converted into a neti pot.

An advantage of an embodiment of the invention is to provide a nasal cleansing top that quickly and easily coverts a vessel such as a nasal rinse bottle into a neti pot.

An advantage of an embodiment of the invention is to provide an improved nasal cleansing dispenser head that helps overcome the disadvantages discussed above.

An advantage of an embodiment of the invention is to provide a nasal cleansing dispenser head that can be easily handled, is convenient to use, and that allows cleansing liquid to flow at various angles.

An advantage of an embodiment of the invention is to provide an inexpensive and well functioning nasal cleansing dispenser head for multiple use.

An advantage of an embodiment of the invention is to provide a readily and inexpensively manufactured nasal cleansing dispenser head.

An advantage of an embodiment of the invention can be obtained, at least in part, via a hollow nasal cleansing top or dispenser head having a curved shape that tapers from a base portion of the head to a tip portion of the head. The dispenser head further includes an opening or outlet disposed on a spout end through which a cleansing liquid, such as, for example, saline, can be dispensed.

The dispenser head additionally may include an interface or attachment mechanism that engages a vessel such as a rinse bottle to deliver cleansing saline solution by gravity from the bottle to the nasal cavity.

An embodiment of the present invention is a dispenser head capable of attaching to a vessel containing a nasal rinsing liquid, the dispenser head comprising a body portion having a first end and a second end, a tip disposed at the first end having a first opening, a base portion disposed at the second end having a second opening, and an attachment mechanism disposed at the second opening to removably attach the base portion to the vessel.

The body portion may have a generally conical shape. The body portion may be curved. A cross-section of the body portion may gradually taper along a length from the second end to the first end.

The attachment mechanism may comprise matable threads disposed on the base portion and the vessel, a friction fit between the base portion and the vessel, a twist lock between the base portion and the vessel, or any other suitable attachment mechanism. The liquid from the attached vessel may passe through the dispenser head from the second end to the first end by gravity.

A true angle from a center of the second opening to a center of the first opening may be about 15 to about 35 degrees. An inner curvature of the body portion may be about 30 to about 35 degrees. An outer curvature of the body portion may be about 20 to about 30 degrees.

An embodiment of the present invention is a dispenser head capable of attaching to a vessel containing a nasal rinsing liquid, the dispenser head comprising a body portion having a first end and a second end, a tip disposed at the first end having a first opening, a base portion disposed at the second end having a second opening, an attachment mechanism disposed at the second opening to removably attach the base portion to the vessel, and wherein liquid from the vessel passes through the dispenser head from the second end to the first end by gravity when the dispenser head is attached to the vessel.

Another embodiment of the present invention is a method for cleansing a nasal cavity comprising the steps of: attaching a dispenser head comprising a body portion having a first end and a second end, a tip disposed at the first end having a first opening, a base portion disposed at the second end having a second opening, and an attachment mechanism disposed at the second opening removably attaching the base portion to a vessel containing a nasal cleansing liquid; and releasing the liquid from the vessel through the dispenser head and into the nasal cavity by gravity. The releasing step may be further effected by user hand control.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of the present invention.

### BRIEF DESCRIPTION OF THE DRAWINGS

In order to assist the understanding of this invention, reference will now be made to the appended drawings, in which like reference characters refer to like elements. The drawings are exemplary only, and should not be construed as limiting the invention.

FIG. 1 is a side elevation view of a nasal cleansing dispenser head apparatus suitable for use in the practice of an embodiment of the present invention.

FIG. 2 is a back elevation view of a nasal cleansing dispenser head apparatus suitable for use in the practice of an embodiment of the present invention.

FIG. 3 is an overhead elevation view of the embodiment of FIGS. 1 and 2, of a nasal cleansing dispenser head suitable for use in accordance with an embodiment of the invention.

### DETAILED DESCRIPTION

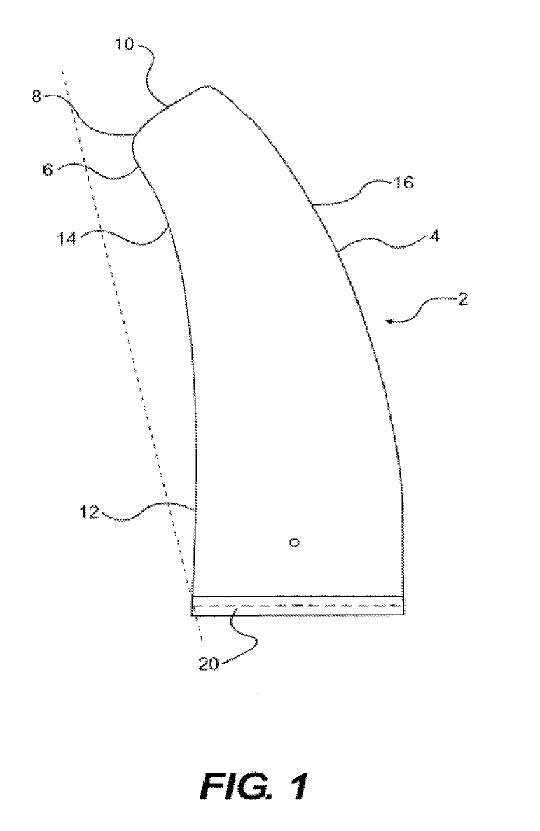

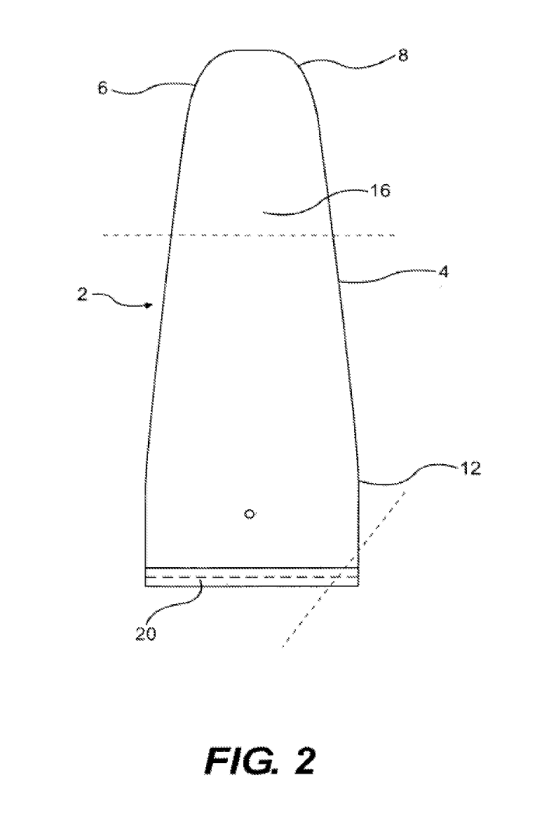

Reference will now be made in detail to embodiments of the present invention, examples of which are illustrated in the accompanying drawings. FIGS. 1 and 2 illustrate nasal cleansing dispenser head 2 in the upright or vertical position. An embodiment of the present invention comprises dispenser head 2 capable of attaching to a vessel, dispenser head 2 comprising body portion 4 having a first end and a second end, tip 6 disposed at the first end, tip 6 having spout end 8 disposed on top surface 18 and having first opening 10, base portion 12 disposed at the second end having a second opening, and attachment mechanism 20 disposed at the second opening to removably attach base portion 12 to the vessel. In particular, an embodiment of the invention contemplates hollow dispenser head 2 having curved body portion 4 leading to smoothly tapered tip 6 at spout end 8 of dispenser head 2.

Curved body portion 4 allows the user to tip an attached vessel up at a variety of angles to flush out the nasal cavities and sinuses. The cleansing solution may be simply poured by gravity, or optionally the vessel may be squeezed to provide a user-controlled pressure flow.

Tip 6 of dispenser head 2 has a generally conical shape, for ease of insertion into the nostrils, and includes opening 10 disposed in top surface 18 of tip 6 for dispensing a liquid.

Opening 10 can have any suitable shape for dispensing a stream of liquid such as, for example, round, oval, square or other suitable geometric shape.

The conical shape of tip 6 provides support for nostril walls of varying sizes, and minimizes spillage by plugging the nostril.

Dispenser head 2 further includes extended base portion 12 having attachment mechanism 20 disposed on an interior surface or in some embodiments on an exterior surface of base portion 12. Attachment mechanism 20 may be in the form of internal threads which are designed to engage the external threads on the circumference of a vessel, external threads to engage the internal threads of a vessel, a friction fit between base portion 12 of dispenser head 2 and the vessel, a twist lock mechanism wherein a pin engages a slot and upon exerting a twisting motion removably locks the pin in the slot, or any other suitable and/or equivalent attachment mechanism (not shown). In an embodiment, attachment mechanism 20 may further comprise a sealing mechanism, for example, an o-ring (not shown). These and other attachment mechanisms are well known to those of ordinary skill in the art and are deemed disclosed herein.

Attachment mechanism 20 allows dispenser head 2 to removably attach to a vessel, for example, to the neck of a rinse bottle, including, but not limited to, a nasal rinse bottle, without the risk of easy separation from the vessel when dispenser head 2 is attached to the vessel and the assembled unit is in use.

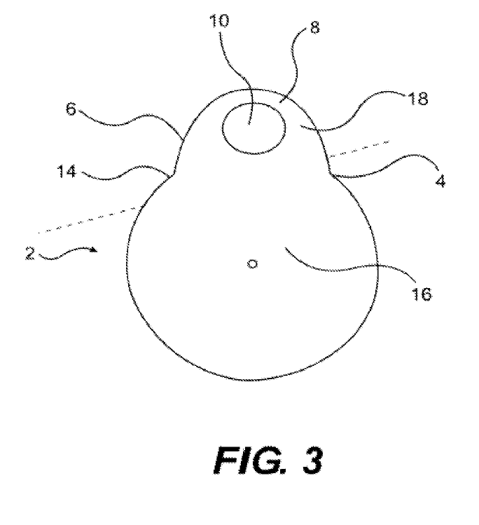

FIG. 3 is an overhead view of dispenser head 2, showing first opening 10 formed in top surface 18 of spout end 8 of tip 6 as well as curved body portion 4 of dispenser head 2.

The true angle following the middle of curved body portion 4 from the second end to the first end comprising spout end 8 and opening 10 can be about 15 degrees to about 35 degrees, about 18 degrees to about 32 degrees, or, in accordance with certain embodiments of the present invention, about 25 degrees.

In accordance with an embodiment, body portion 4 of dispenser head 2 can have inner curvature 14 of about 30 degrees to about 35 degrees and outer curvature 16 of about 20 degrees to about 30 degrees from the second end to the first end.

In an embodiment, dispenser head 2 is made of a plastic material (e.g., polypropylene, polyethylene, polyvinyl chloride, or the like) or a metal material (e.g., aluminum, magnesium, or the like), or any other suitable material. Dispenser head 2 may be adapted or fitted to any plastic, metal or glass vessel for nasal cleansing.

Although the invention has been described as a nasal cleansing dispenser head, it will be apparent to those skilled in the art that the embodiments disclosed herein are amenable to modifications, such as alternative materials, which can render the device suitable for diverse variations without departing from the scope or spirit of the invention or adversely affecting the operability of same. Thus, it is intended that the present invention cover all such modifications and variations of the invention, provided they come within the scope of the appended claims and their equivalents.

I claim:

1. A dispenser head capable of attaching to a vessel containing a nasal rinsing liquid, the dispenser head comprising:
a body portion having a first end and a second end;
a tip disposed at the first end having a first opening;
a base portion disposed at the second end having a second opening; and
an attachment mechanism disposed at the second opening to removably attach the base portion to the vessel.

2. The dispenser head of claim 1, wherein the body portion has a generally conical shape.

3. The dispenser head of claim 1, wherein the body portion is curved.

4. The dispenser head of claim 1, wherein a cross-section of the body portion gradually tapers along a length from the second end to the first end.

5. The dispenser head of claim 1, wherein the attachment mechanism comprises matable threads disposed on the base portion and the vessel.

6. The dispenser head of claim 1, wherein the attachment mechanism comprises a friction fit between the base portion and the vessel.

7. The dispenser head of claim 1, wherein the attachment mechanism comprises a twist lock between the base portion and the vessel.

8. The dispenser head of claim 1, wherein liquid from the vessel passes through the dispenser head from the second end to the first end by gravity.

9. The dispenser head of claim 1, wherein a true angle from a center of the second opening to a center of the first opening is about 15 to about 35 degrees.

10. The dispenser head of claim 1, wherein an inner curvature of the body portion is about 30 to about 35 degrees.

11. The dispenser head of claim 1, wherein an outer curvature of the body portion is about 20 to about 30 degrees.

12. A dispenser head capable of attaching to a vessel containing a nasal rinsing liquid, the dispenser head comprising:
a body portion having a first end and a second end;
a tip disposed at the first end having a first opening;
a base portion disposed at the second end having a second opening;
an attachment mechanism disposed at the second opening to removably attach the base portion to the vessel; and
wherein liquid from the vessel passes through the dispenser head from the second end to the first end by gravity when the dispenser head is attached to the vessel.

13. The dispenser head of claim 12, wherein the body portion has a generally conical shape and is curved.

14. The dispenser head of claim 12, wherein a true angle from a center of the second opening to a center of the first opening is about 15 to about 35 degrees.

15. A method for cleansing a nasal cavity comprising the steps of:
attaching a dispenser head comprising a body portion having a first end and a second end;
a tip disposed at the first end having a first opening;
a base portion disposed at the second end having a second opening; and
an attachment mechanism disposed at the second opening removably attaching the base portion to a vessel containing a nasal cleansing liquid; and
releasing the liquid from the vessel through the dispenser head and into the nasal cavity by gravity.

16. The method of claim 15, wherein the dispenser head body portion has a generally conical shape.

17. The method of claim 15, wherein the dispenser head body portion is curved.

18. The method of claim 15, wherein a cross-section of the dispenser head body portion gradually tapers along a length from the second end to the first end.

19. The method of claim 15, wherein a true angle from a center of the second opening to a center of the first opening is about 15 to about 35 degrees.

20. The method of claim 15, wherein the releasing is further effected by user hand control.
